(19) United States
(12) Patent Application Publication
(10) Pub. No.: US 2010/0040545 A1
(43) Pub. Date: Feb. 18, 2010

(54) RADIOLABELLING METHODS

(76) Inventors: Erik Arstad, London (GB); David Turton, London (GB)

Correspondence Address: GE HEALTHCARE, INC., IP DEPARTMENT 101 CARNEGIE CENTER, PRINCETON, NJ, 08540-6231, US

(21) Appl. No.: 12/519,797

(22) PCT Filed: Dec. 19, 2007
(86) PCT No.: PCT/GB2007/004884

§ 371 Date: Jun. 18, 2009

Related U.S. Application Data
(60) Provisional application No. 60/871,140, filed on Dec. 21, 2006.

(51) Int. Cl. A61K 51/04; C07B 59/00; C07D 265/34; C07F 7/08; C07D 217/04; C07D 221/18

(52) U.S. Cl. 424/1.81; 546/146; 546/150; 544/69; 544/101; 546/75

(57) ABSTRACT

The present invention relates to methods of synthesising radiolabelled compounds, to the precursors useful in such methods and to the radiolabelled compounds obtainable by such methods. More particularly, the present invention relate to methods, precursors and radiolabelled compounds useful in Positron Emission Tomography (PET) and Single Photon Emission Computed Tomography (SPECT) especially for imaging neuroreceptors with radiolabelled agonists.

The present invention relates to methods of synthesising radiolabelled compounds, to the precursors useful in such methods and to the radiolabelled compounds obtainable by such methods. More particularly, the present invention relates to methods, precursors and radiolabelled compounds useful in Positron Emission Tomography (PET) and Single Photon Emission Computed Tomography (SPECT) especially for imaging neuroreceptors with radiolabelled agonists.

Radiolabelled amines are of interest for PET imaging in general and in particular for imaging neuroreceptors with radiolabelled agonists.

It has been proposed in the ternary complex model that, in vivo, guanidine nucleotide-coupled dopamine subtype 2 receptors (D₂) are configured in high and low affinity states for the dopamine agonist. It is thought that D₂ agonists bind with high affinity to high affinity states (D_{2 high}) and with low affinity to low affinity states (D_{2 low}) in contrast to D₂ antagonists (for example, [¹¹C]raclopride) which will bind with equal affinity to the two states. In consequence, an agonist tracer should be an effective probe of D_{2 high} receptors in vivo. Additionally, because dopamine itself binds well to D_{2 high} states, an agonist tracer will be particularly sensitive to endogenous dopamine concentration changes.

Known D₂ agonists include apomorphine, aminotetralin derivates, and (+)-4-propyl-3,4,4a,5,6,10b-hexahydro-2H-naphtho[1,2-b][1,4]oxazin-9-ol (PHNO).

These agonists are discussed in Hwang et al Bioconjugate Chemistry 2005 16 pp 27-31, together with disclosure of radiolabelling methods for radiolabelling promising agonists.

Radiolabelled PHNO is also discussed in Wilson et al J. Med. Chem. 2005 48 pp 4153-4160 together with discussion of a protocol for the radiosynthesis of [¹¹C] PHNO.

The apomorphine derivative (−)-N-propyl-norapomorphine (NPA) radiolabelled with ¹¹C is discussed in Hwang et al Nuclear Medicine and Biology Vol 27 (2000) pp 533-539.

Radionuclides suitable for use in tracers for PET and SPECT often have short half-lives. For example, two useful radionuclides ¹¹C and ¹⁸F have half-lives of about 20 and 110 minutes respectively. A result of this is that it is of utmost importance that protocols for radiolabelling tracer compounds have as few steps as possible, and are both convenient and quick, with as high yields as possible.

Unfortunately, usual methods of radiolabelling the compounds discussed above are time consuming. For example, current methods for labelling NPA are only capable of providing low yields in a multistep procedure requiring the formation of a carboxylic acid, subsequent transformation to an acyl chloride followed by reaction with an amine precursor, reduction of the corresponding amide and finally deprotection.

The present invention aims to address this problem.

The present invention accordingly provides, in a first aspect, a method for synthesising a radiolabelled compound, the method comprising reacting a compound of formula I:

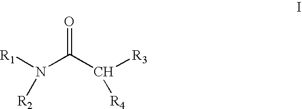

with a compound containing a radionuclide;
in the presence of a base;
wherein R₁ and R₂:
a) are independently selected from hydrocarbyl and heterohydrocarbyl; or,
b) together with the nitrogen atom to which they are attached form a nitrogen-containing heterohydrocarbyl ring; and,
   R₃ and R₄ are independently selected from hydrogen, hydrocarbyl and heterohydrocarbyl.

The method is advantageous because it is simple and enables radiolabelling with fewer steps than currently used, increasing yield and reducing the time required to synthesise the labelled compound. The precursor of formula I is particularly advantageous since, as an acetamide it is relatively easy to synthesise (e.g. by treating the corresponding amine with acyl chloride), and is unlikely to suffer from poor stability

Preferably the radionuclide is selected from ¹¹C, ¹⁸F, ⁷⁵Br, ⁷⁶Br, and ¹²⁴I. The more preferred radionuclides are ¹¹C or ¹⁸F. If ⁷⁵Br, ⁷⁶Br, or ¹²⁴I are used they should, preferably, be used as a substituent on an aromatic fragment.

In a preferred embodiment of the method the compound containing a radionuclide is of formula R*X, wherein R* is hydrocarbyl containing the radionuclide and X is a leaving group.

Advantageously R* is a radiolabelled alkyl group, especially a C₁-C₆ alkyl group and most preferably selected from a radiolabelled methyl, ethyl, propyl, isopropyl, butyl, isobutyl and t-butyl group.

The R* fragment may be labelled with ¹⁸F (e.g. [¹⁸F]C₁-C₆ alkyl, in particular ¹⁸FCH₂—) but is preferably labelled with ¹¹C.

X may be any leaving group of sufficient lability and stability to work in the method. If the radionuclide is ¹⁸F, the leaving group should have higher lability than F⁻.

The preferred leaving groups are sulfonate leaving groups or—halogen. Preferred sulfonate leaving groups are triflate, mesylate, tosylate or besylate. Preferred halogens are chloro, bromo or iodo. The most preferred leaving groups are iodo or triflate.

In the most preferred embodiment of the method, the compound containing the radionuclide is ¹¹CH₃I. This is advantageous because [¹¹C] iodo methane is convenient to prepare and to use.

The compound of formula I is reacted in the presence of a base, resulting in substitution of the α hydrogen of the compound with the species containing a radionuclide (e.g. R*).

Suitable bases include lithium bis(trimethylsilyl)amide (LHMDS, usually used in tetrahydrofuran —THF— solution).

For the synthesis of agonists, the method preferably includes a further step of reduction of the labelled amide to amine. Suitable reducing agents include lithium aluminium hydride.

The overall method is simple, convenient and has the effect of reducing the number of steps required to provide a radiolabelled agonist (including reduction and deprotection) to two since the reduction step (e.g. when using LiAlH₄ as the reducing agent) can result in reduction and deprotection of the alcohol protecting group in one step. This is greatly advantageous especially when using ¹¹C or ¹⁸F with short half-lives. Another advantage is that the method is suitable for one-pot procedures which make it possible to use commercially available synthesis modules (e.g. in a clinical environment such as a hospital). Previously used methods with a greater number of steps and involving a more complex protocol required specially made synthesis modules increasing the cost and difficulty of the process.

Examples of suitable “alcohol protecting groups” are: methyl, ethyl or tert-butyl; alkoxymethyl or alkoxyethyl; benzyl; acetyl; benzoyl; trityl (Trt) or trialkylsilyl such as tetrabutyldimethylsilyl.

R₃ and R₄ are preferably hydrogen, but may be independently selected from C₁-C₆ alkyl, preferably C₁ to C₄ alkyl. If R₃ and R₄ are independently C₁ to C₄ alkyl, it is preferred if they are straight chain alkyl for example methyl, ethyl, n-propyl or n-butyl.

R₂ may be selected from alkyl, aryl and alkylaryl, in particular n-propyl F—(CH₂)₅—, —CH₂CH₂C₆H₅ or —CH₂CH₂C₆H₁₁, and R₁ is preferably

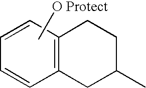

wherein —O Protect is an alcohol protecting group. More preferably R₁ is:

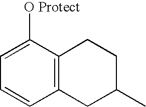

Reduction and deprotection of the alcohol protecting group would give an aminotetralin useful as an agonist.

Alternatively, R₁ and R₂ together with the nitrogen to which they are attached may form:

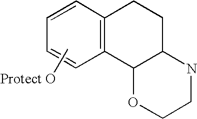

wherein —O Protect is a alcohol protecting group. Reduction and deprotection would give in this case (when R*X is ¹¹CH₃I in the method) PHNO, also a useful agonist.

A further alternative is that R₁ and R₂ together with the nitrogen to which they are attached may form:

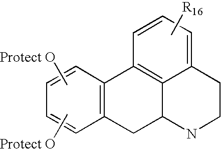

wherein —O Protect is an alcohol protecting group. Reduction and deprotection would give NPA, a useful agonist.

In an alternative embodiment, R₁ and R₂ together form a five or six member ring.

As discussed above in relation to the first aspect of the invention, the acetamide precursor is advantageous because it has good stability (e.g. on storage) and is relatively simple to prepare.

The present invention accordingly provides, in a second aspect, a compound of formula II:

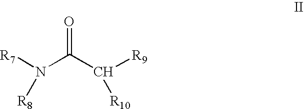

wherein R₉ and R₁₀ are independently selected from hydrogen, hydrocarbyl and heterohydrocarbyl; and R₇ and R₈ are as defined in (a), (b), or (c), below:
a) R₇ is

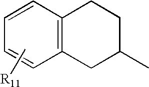

and R₈ is selected from hydrocarbyl and heterohydrocarbyl;
b) R₇ and R₈ together with the nitrogen to which they are attached form

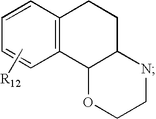

or
c) R₇ and R₈ together with the nitrogen to which they are attached form

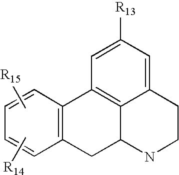

wherein each of R₁₁, R₁₂, R₁₃, R₁₄ and R₁₅ are independently selected from hydrogen, hydroxyl, alkoxyl and -[alcohol protecting group];
with the proviso that when R₇ and R₈ are as defined in (b) neither R₉ nor R₁₀ are methyl.
Preferably, an additional proviso in the definition of formula II is when R₇ and R₈ are as defined in (c) none of R₁₃-R₁₅ is hydrogen.

Preferably, at least one of R₉ and R₁₀ is hydrogen, more preferably both R₉ and R₁₀ are hydrogen. R₉ and R₁₀ may alternatively be independently selected from C₁ to C₆ alkyl, in particular C₁ to C₄ alkyl preferably methyl, ethyl, n-propyl or n-butyl.

As discussed above, the acetamide precursor may conveniently be prepared by treating the corresponding amine with acetyl chloride.

In the more preferred embodiments of the second aspect of the invention, the compound according to formula II is of formula

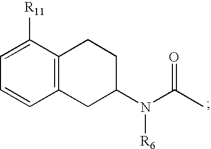

of formula

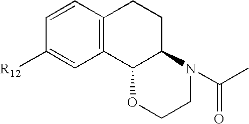

or, of formula

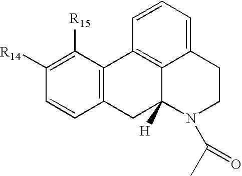

The product of the method of the first aspect of the present invention when ¹¹CH₃I (or another [¹¹C]-methyl reagent) is used and after reduction of the amide is a propylamine.

The present invention, accordingly provides, in a third aspect, a compound of formula III:

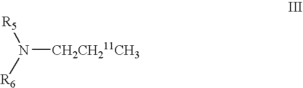

wherein R₅ and R₆ are:
a) independently selected from hydrocarbyl or heterohydrocarbyl; or
b) together with the nitrogen atom to which they are attached form a five or six-member ring.

### R₅ may be:

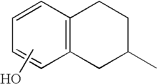

With —OH in the 5 position the compound would be of formula:

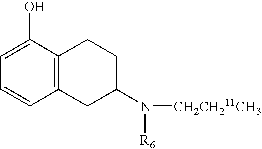

Alternatively, R₅ and R₆ together with the nitrogen to which they are attached may form:

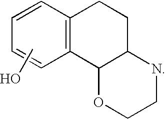

In which case, the compound may be of formula:

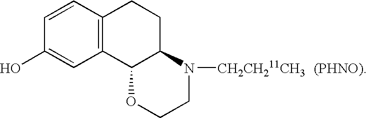

Alternatively, R₅ and R₆ together with the nitrogen to which they are attached may form:

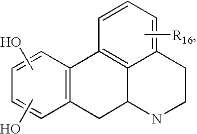

wherein R₁₆ is selected from hydrogen, hydroxyl, and alkoxyl. In which case, the compound may be of formula:

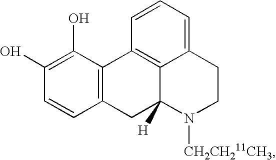

### NPA.

In this specification, unless otherwise specified, “hydrocarbyl” refers to an optionally substituted hydrocarbon group and includes alkyl, alkenyl, alkynyl, 5- or 6-membered rings (that may be alicyclic or aryl and includes monocyclic, bicyclic or polycyclic fused ring systems), preferably C₁ to C₃₂, more preferably C₁ to C₂₄, most preferably C₁ to C₁₈.

“Heterohydrocarbyl” refers to a group as defined above for hydrocarbyl but containing one or more heteroatoms preferably selected from N, O and S.

Alkyl is preferably C₁ to C₆, more preferably straight chain C₁ to C₆ in particular methyl, ethyl, n-propyl or n-butyl.

Also provided by the present invention is a radiopharmaceutical composition comprising the compound of formula III as defined above; together with a biocompatible carrier.
The “biocompatible carrier” is a fluid, especially a liquid, in which the compound of formula III is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.
A further aspect of the present invention is a compound of formula III as defined above for medical use. Preferably, said medical use is a method for the diagnosis of a condition in which the expression of high affinity dopamine subtype 2 receptors (D_{2 high}) is perturbed. For example, the availability of these receptors has been shown to be reduced vs. normal in the temporal cortex in Alzheimer's disease (Joyce et al 1998 Brain Research 784: 7-17) and in the striatum in Huntington's disease (van Oostrom et al 2005 Neurology 65: 941-3), but increased in the putamen contralateral to the predominant symptoms, compared with the ipsilateral putamen, in early Parkinson's disease (Kassinen et al 2000 J. Nuc. Med. 41: 65-70). Such medical use preferably comprises a method of generating an image of a human or animal body comprising:
(i) providing a subject to whom a detectable quantity of the radiopharmaceutical composition of the invention has been administered;
(ii) allowing the radiopharmaceutical composition to bind to high affinity dopamine subtype 2 receptors (D_{2 high}) in said subject;
(iii) detection of signals emitted by said radiopharmaceutical composition by PET; and,
(iv) generation of an image representative of the location and/or amount of said signals.
   The method of generating an image begins by “providing” a subject to whom a detectable quantity of the radiopharmaceutical composition of the invention has been administered. The purpose of the method of the invention is the provision of a diagnostically-useful image. Therefore, administration to the subject of the radiopharmaceutical composition can be understood to be a preliminary step necessary for facilitating generation of said image. Preferably said subject is a mammal, and most preferably a human. Most preferably, said subject is the intact mammalian body in vivo. A preferred route of administration is intravascular administration. In an alternative embodiment, administration of a detectable quantity of the radiopharmaceutical composition may be carried out as part of the method of the invention.
   Following the providing step and preceding the detection step, the radiopharmaceutical composition is allowed to bind to D_{2 high} in said subject. For example, when the subject is an intact mammal, the radiopharmaceutical composition will dynamically move through the mammal's body, coming into contact with various tissues therein. Once the radiopharmaceutical composition comes into contact with any D_{2 high}, a specific interaction takes place such that clearance of the radiopharmaceutical composition from tissue expressing D_{2 high} takes longer than from non-expressing tissue. A certain point in time will be reached when detection of radiopharmaceutical composition specifically bound to tissue expressing D_{2 high} is enabled as a result of the ratio between radiopharmaceutical composition bound to tissue expressing D_{2 high} versus that bound in non-expressing tissue. An ideal such ratio is at least 2:1.
   The “detection” step of the method of the invention involves the detection of signals emitted by the ¹¹C of the radiopharmaceutical composition by means of a detector sensitive to said signals. This detection step can also be understood as the acquisition of signal data. The “generation” step of the method of the invention is carried out by a computer which applies a reconstruction algorithm to the acquired signal data to yield a dataset. This dataset is then manipulated to generate images showing areas of interest within the subject.

In a yet further aspect, the present invention provides for use of a compound of formula III as defined above for the manufacture of a radiopharmaceutical for use in the diagnosis of a condition in which the expression of high affinity dopamine subtype 2 receptors (D_{2 high}) is perturbed.

The invention will now be illustrated by the following non-limiting examples.

### EXAMPLE 1

The synthesis of [¹¹C]NPA according to the method of the invention is illustrated in Scheme 1.

### EXAMPLE 2

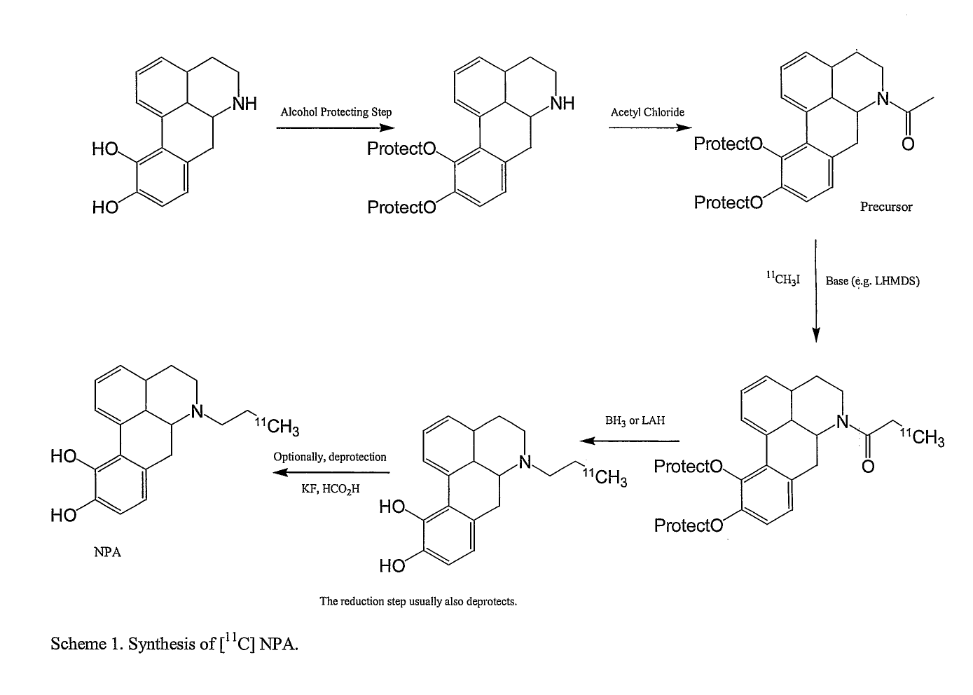

### Radiosynthesis of [¹¹C]—N-propionyl-1,2,3,4-tetrahydroisoquinoline (1)

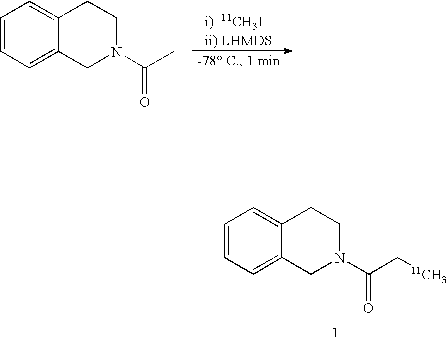

[¹¹C]Methyl iodide was distilled over a closed vial containing N-acetyl-1,2,3,4-tetrahydroisoquinoline (5-25 μmol) dissolved in THF (0.2-0.3 ml). The reaction vial was cooled down to −78° C. using an acetone/dry ice bath. 1 M lithium bis-(trimethylsilyl)amide (LHMDS) solution in THF (1 equivalent) was added and after 1 minute the reaction mixture was quenched with 0.1 ml of acetic acid 0.1 M in methanol followed by dilution with HPLC mobile phase (70% water containing 0.1% TFA-30% acetonitrile containing 0.1% TFA). The samples were analysed in a Phenomenex Luna C18(2) column (150 mm3 4.6 mm 35 micron), 1 ml/min, wavelength=254 nm. HPLC radiochemical yields were 92-98% (FIG. 1).

### Radiosynthesis of [¹¹C]—N-propyl-1,2,3,4-tetrahydroisoquinoline (2) through the Sep-Pak method

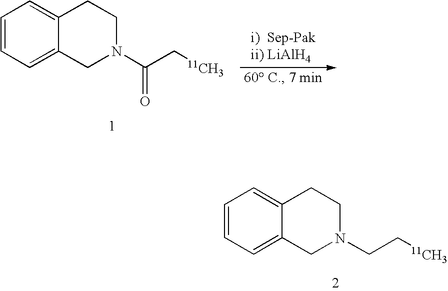

[¹¹C]—N-Propionyl-1,2,3,4-tetrahydroisoquinoline (1) was prepared as described above. The reaction mixture was passed through a light silica Sep-Pak preconditioned with THF (15 ml). 0.4-0.5 ml of extra THF were used to elute the radioactive product (1). LiAlH₄ (1 M in THF, 7 equivalents) was added to the eluate and the reaction was heated to 60° C. for 7 minutes. Samples were quenched with 0.1 ml of aqueous sodium hydroxide (10% w/w) and diluted with 0.2 ml of HPLC mobile phase (50% (NH₄)₂HPO₄ 0.05 M-50% acetonitrile). The samples were analysed in a Phenomenex Luna C18(2) column (150 mm3 4.6 mm 35 micron), 1 ml/min, wavelength=254 nm. HPLC radiochemical yields were 75-95% (FIG. 2).

### One-pot radiosynthesis of [¹¹C]—N-propyl-1,2,3,4-tetrahydroisoquinoline (2)

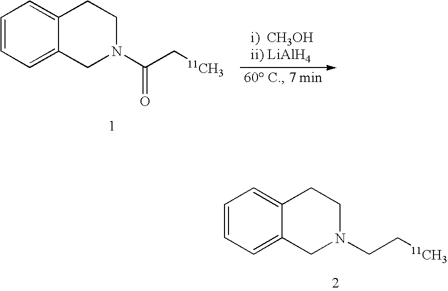

[¹¹C]—N-Propionyl-1,2,3,4-tetrahydroisoquinoline (1) was prepared as described above. The reaction was quenched with anhydrous methanol (2.5 equivalents) and was warmed up to room temperature. LiAlH₄ (1 M in THF, 9 equivalents) was added and the reaction was heated to 60° C. for 7 minutes. Samples were quenched with 0.1 ml of aqueous sodium hydroxide (10% w/w) and diluted with 0.2 ml of HPLC mobile phase (50% (NH₄)₂HPO₄ 0.05 M-50% acetonitrile). The samples were analysed in a Phenomenex Luna C18(2) column (150 mm3 4.6 mm 35 micron), 1 ml/min, wavelength=254 nm. HPLC radiochemical yields were 75-77% (FIG. 3).

### EXAMPLE 3

### Radiosynthesis of [¹¹C]-(+)-4-Propionyl-3,4,4a,5,6,10b-hexahydro-9-triisopropylsilyloxy-2H-naphtho[1,2-b][1,4]oxazine (4)

### Procedure 1:

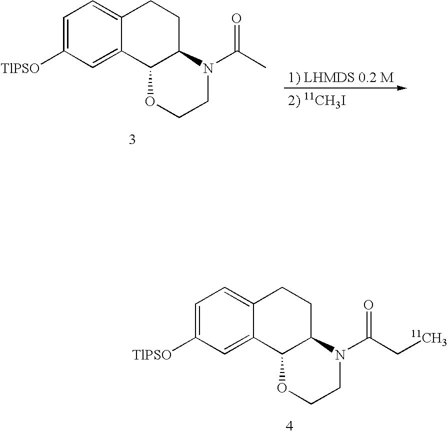

The acetyl precursor (3)(2-3 mg, 5-7.5 μmol) was dissolved in 100 μl of THF (stirring needed). The solution was kept in an acetone/dry ice bath (−78° C.) and a solution of lithium bis-(trimethylsilyl)amide (LHMDS, 0.2 M in THF, 2.2-3 equivalents relative to the acetyl precursor 3, 55-112 μl) was added dropwise. The reaction mixture (slightly yellow solution) was allowed to warm up for 3-7 minutes and 25 μl of ¹¹CH₃I/THF were added. The reaction was completed in less than 5 minutes (no trace of ¹¹CH₃I observed). Vials containing 0.2 ml of mobile phase and 0.1 ml of acetic acid (0.2 M in methanol) were used to quench analytical samples (10-20 μl).

### Procedure 2:

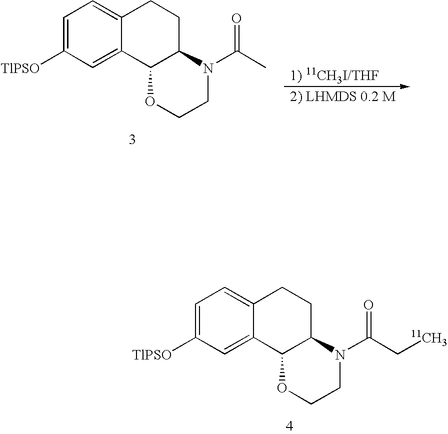

The acetyl precursor (3)(2-3 mg, 5-7.5 μmol) was dissolved in 150 μl of ¹¹CH₃I/THF solution at room temperature (stirring needed). LHMDS (0.2 M in THF, 2.2 equivalents, 55-82 μl) was added dropwise at room temperature and the reaction was completed in less than 5 minutes (no trace of ¹¹CH₃I observed). Vials containing 0.2 ml of mobile phase and 0.1 ml of acetic acid (0.2 M in methanol) were used to quench analytical samples (10-20 μl).

### HPLC Conditions:

Phenomenex Luna 3μ C18(2), 50 3 4.6 mm, 100 Å. Wavelength=280 nm, 1 ml/min. Mobile phase: 20% water (with 0.1% TFA)-80% acetonitrile (with 0.1% TFA).

### Retention Times:

Acetyl precursor 3 (UV): 4.5-4.7 minutes.
Carbon-11 product 4 (radioactivity): 6.4-6.7 minutes.
Unknown radioactive product: 0.7-0.9 minutes.
Carbon-11 methyl iodide: 1.3-1.4 minutes.
HPLC radiochemical yields for 4 were 50-85% (FIG. 1).

### Notes:

Inhibitor-free anhydrous THF was used in all cases.
Lithium bis-(trimethylsilyl)amide was obtained from Aldrich as a 1 M solution in THF. The 0.2 M solutions of base were prepared in a glove box and could be used at least for 2 days.
The reaction mixture containing 3, THF and LHMDS 0.2 M can be kept at −78° C. up to 30 minutes.

### Radiosynthesis of [¹¹C]-(+)-4-propyl-3,4,4a,5,6,10b-hexahydro-2H-naphtho[1,2-b][1,4]oxazin-9-ol (PHNO, 5)

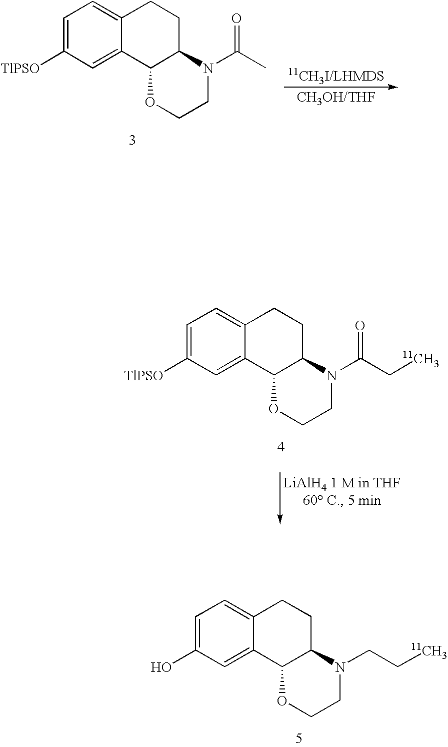

The acetyl precursor (3)(2.6 mg, 6.4 μmol) was dissolved in 100 μl of THF (stirring needed). The solution was kept in an acetone/dry ice bath (−78° C.) and a solution of lithium bis-(trimethylsilyl)amide (LHMDS, 0.2 M in THF, 2.5 equivalents, 80 μl) was added dropwise. The reaction mixture (slightly yellow solution) was allowed to warm up for 3-7 minutes and 25 μl of ¹¹CH₃I/THF were added. The reaction was quenched after 5 minutes with anhydrous methanol (0.2 M solution in THF, 5 equivalents, 160 μl). Lithium aluminium hydride (1 M solution in THF, 15 equivalents, 96 μl) was added dropwise. The reaction vial needed to be vented during this addition due to hydrogen formation. After the LiAlH₄ addition, the reaction vial was heated at 60° C. for 5 minutes (no vent required). Vials containing 0.2 ml of mobile phase and two drops of acetic acid were used to quench analytical samples (10 μl).

### HPLC Conditions:

Semi-preparative Phenomenex Luna 10μ C18(2), 250 3 10 mm, 100 Å. Wavelength=280 nm, 3 ml/min. Mobile phase: water/0.1 M ammonium formate (adjusted to pH 4.5 with acetic acid)-acetonitrile.
A gradient method was used:
0-1 minutes: 25% acetonitrile
1-6 minutes: from 25% to 45% acetonitrile
6-10 minutes: from 45% to 95% acetonitrile
10-30 minutes: 95% acetonitrile
30-35 minutes: back to 25% acetonitrile

### Retention Times:

4-Ethyl-3,4,4a,5,6,10b-hexahydro-2H-naphtho[1,2-b][1,4]oxazin-9-ol (reduced and deprotected precursor, UV): 6.2-6.4 minutes.
Carbon-11 PHNO 5 (radioactivity): 7.9-8.1 minutes.
Unknown radioactive products: 4.1 minutes, 4.7 minutes and 6.7 minutes.
Methyl iodide: 13.7 minutes.
Cold precursor 3: 25.8 minutes.
HPLC radiochemical yield for 5 was 60% (FIG. 2).

### Notes:

The radio-HPLC peak areas after carbon-11 methylation and after reduction-deprotection should not vary much, i.e. an experiment with 60% yield of 4 should have around 60% yield of 5.
Cold experiments showed that the reduction-deprotection happened after 3 minutes, so the 5 minutes used in radiochemistry can probably be reduced.

### Quenching and Solubilisation of Lithium Aluminium Hydride:

Starting with 50 μl of LiAlH₄ (1 M solution in THF) and 0.2 ml THF:
Acidic conditions: Addition of 0.5-0.6 ml 0.5 M HCl gave a clear solution.
Basic conditions: Clear solutions were obtained adding 2 ml NaOH 1.5 M, EDTA disodium salt (0.125 M, 2 ml)+0.5 ml NaOH 6.25 M, EDTA trisodium salt (0.1 M, 2 ml)+0.3 ml NaOH 6.25 M.
Smaller volumes of reagents may be required if THF is evaporated.

What is claimed is:

1.-35. (canceled)

36. A method for synthesising a radiolabelled compound, the method comprising reacting a compound of formula I: 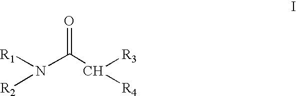
with a compound containing a radionuclide of formula R*X wherein R* is a ¹¹C-labelled alkyl group selected from a ¹¹C-labelled methyl, ethyl, propyl, isopropyl, butyl, isobutyl and t-butyl and X is a leaving group;
in the presence of a base;
wherein R₁ and R₂:
a) are independently selected from hydrocarbyl and heterohydrocarbyl; or,
b) together with the nitrogen atom to which they are attached form a nitrogen-containing heterohydrocarbyl ring; and,
R₃ and R₄ are independently selected from hydrogen, hydrocarbyl and heterohydrocarbyl.

37. A method as claimed in claim 36, wherein X is selected from chloro, bromo, iodo and triflate.

38. A method as claimed in claim 36 wherein the compound containing the radionuclide is ¹¹CH₃I.

39. A method as claimed in claim 36 wherein R₃ and R₄ are independently selected from hydrogen and C₁ to C₆ alkyl.

40. A method as claimed in claim 36, wherein R₂ is selected from alkyl, aryl, and alkylaryl.

41. A method as claimed in claim 40, wherein R₁ is: 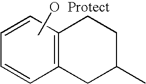
wherein —O Protect is an alcohol protecting group.

42. A method as claimed in claim 36 wherein R₁ and R₂ together with the nitrogen to which they are attached form: 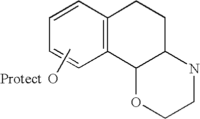
wherein —O Protect is a alcohol protecting group.

43. A method as claimed in claim 36, wherein R₁ and R₂ together with the nitrogen to which they are attached form: 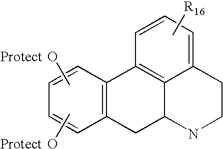
wherein R₁₆ is selected from hydrogen, hydroxyl, alkoxyl and —O Protect;
wherein —O Protect is an alcohol protecting group.

44. A compound of formula II: 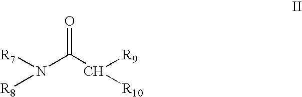
wherein R₉ and R₁₀ are independently selected from hydrogen, hydrocarbyl and heterohydrocarbyl; and R₇ and R₈ are as defined in (a), (b), or (c), below:
(a) R₇ is 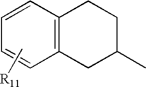
and R₈ is selected from hydrocarbyl and heterohydrocarbyl;
(b) R₇ and R₈ together with the nitrogen to which they are attached form: 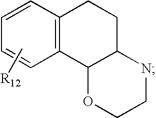
or
(c) R₇ and R₈ together with the nitrogen to which they are attached form: 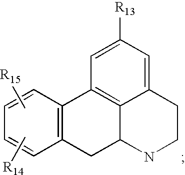
wherein each of R₁₁, R₁₂, R₁₃, R₁₄ and R₁₅ are independently selected from hydrogen, hydroxyl, alkoxyl and -[alcohol protecting group];
with the proviso that when R₇ and R₈ are as defined in (b) neither R₉ nor R₁₀ are methyl and with the additional proviso that when R₇ and R₈ are as defined in (c) none of R₁₃-R₁₅ is hydrogen.

45. A compound as claimed in claim 44, wherein R₉ and R₁₀ are independently selected from hydrogen and C₁ to C₆ alkyl.

46. A compound of formula III: 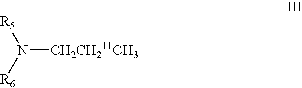
wherein R₅ and R₆ are:
(a) independently selected from hydrocarbyl or heterohydrocarbyl; or,
(b) together with the nitrogen atom to which they are attached form a five or six-member ring.

47. A compound as claimed in claim 46, of formula:

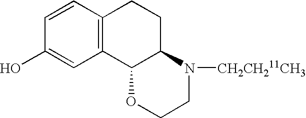

48. A compound as claimed in claim 46 of formula:

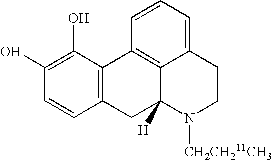

49. A radiopharmaceutical composition comprising the compound according to claim 36; together with a biocompatible carrier.

50. A compound according to claim 46 for medical use.
